(19) United States
(12) Patent Application Publication
(10) Pub. No.: US 2010/0015717 A1
(43) Pub. Date: Jan. 21, 2010

(54) Mass Tags for Quantitive Analysis

(75) Inventors: Rajendra Singh, San Jose, CA (US); Haihong Zhou, Mountain View, CA (US)

Correspondence Address: SHERIDAN ROSS PC, 1560 BROADWAY, SUITE 1200, DENVER, CO, 80202, US

(73) Assignee: PPD BIOMARKER DISCOVERY SCIENCES LLC, Wilmington, NC (US)

(21) Appl. No.: 12/391,119

(22) Filed: Feb. 23, 2009

Related U.S. Application Data
(63) Continuation of application No. 12/172,952, filed on Jul. 14, 2008, now abandoned.
Application No. 12/172,952 is a continuation of application No. 11/220,746, filed on Sep. 6, 2005, now abandoned.
Application No. 11/220,746 is a continuation of application No. 10/047,434, filed on Oct. 25, 2001, now abandoned.
(60) Provisional application No. 60/306,747, filed on Jul. 19, 2001.
(60) Provisional application No. 60/296,064, filed on Jun. 5, 2001.
(60) Provisional application No. 60/295,386, filed on May 31, 2001.
(60) Provisional application No. 60/243,394, filed on Oct. 25, 2000.

(51) Int. Cl. G01N 33/68; C07K 2/00; C07D 235/02; C07D 401/00; C07D 409/00

(52) U.S. Cl. 436/86; 530/300; 548/304.1; 546/273.7; 548/962

(57) ABSTRACT

The present invention relates generally to novel protein modification reagents for fractionation and quantitative (differential) profiling of proteins in a complex mixture. The reagents react with amino acids or other protein components or structures and function as mass tags. The present invention provides methods of making the protein modification reagents and methods of using the protein modification reagents for quantitative analysis of proteins.

### RELATED APPLICATIONS

This application is a continuation application of U.S. patent application Ser. No. 12/172,952, filed Jul. 14, 2008, which is a continuation application of U.S. patent application Ser. No. 11/220,746, filed Sep. 6, 2005, which is a continuation application of U.S. patent application Ser. No. 10/047,434, filed on Oct. 25, 2001, which claims the benefit of U.S. Provisional Application Ser. No. 60/243,394, filed on Oct. 25, 2000, U.S. Provisional Application Ser. No. 60/295,386, filed on May 31, 2001, U.S. Provisional Application Ser. No. 60/296,064, filed on Jun. 5, 2001, and U.S. Provisional Application Ser. No. 60/306,747, filed on Jul. 19, 2001, each of which is incorporated herein by reference in its entirety.

### FIELD OF THE INVENTION

The present invention relates generally to novel protein modification reagents for fractionation and quantitative (differential) profiling of proteins in a complex mixture.

More particularly, the present invention relates to methods of making the protein modification reagents and methods of using the protein modification reagents for quantitative analysis of proteins.

### BACKGROUND OF THE INVENTION

Proteomics is the large-scale study of proteins, usually by biochemical methods. Traditionally, proteome analysis is accomplished by a combination of two dimensional gel electrophoresis to separate and visualize proteins and mass spectrometry (MS) for protein identification. Although mass spectrometry is unparalleled in its ability to characterize proteins, it requires significant sample preparation to simplify complex protein mixtures and is an inherently qualitative method that is deficient for quantitative profiling.

There is an unmet need for proteomic technologies that enable comprehensive biomarker and target discovery for detection, prognosis, patient stratification, and therapeutics. Revolutionary advances in genomics technologies have lead to sequencing of the entire human genome and have enabled SNP mapping, and DNA-based genomic profiling at unprecedented high throughput. The need to understand biology at a systems level and to discover disease biomarkers similarly demands a comprehensive interrogation of the proteome. Proteins are, after all, the active agents of expressed genes, the expressed biomarkers reflecting both genetic and environmental influences, and the target of most therapeutic agents. By comparison to genomics, however, efforts to analyze proteins from cells and extracellular spaces on a global scale are still in active development. The intelligent integration of data relating to expression of proteins, along with expression profiling of mRNA and single nucleotide polymorphisms in DNA, is essential for the understanding of the biology of organisms and the causes of disease. See Pandey et al., Nature, 405: 837-846 (2000).

The analysis of complex protein mixtures involves two basic functions, protein separation and protein detection/quantification. Two-dimensional SDS gel electrophoresis (2-DE) is commonly employed for high-resolution protein separation, but the technique has well recognized limitations when applied to large-scale proteomics. Protein arrays, either on chips or with self-encoded elements in solution, may surpass 2-DE as the next generation proteomics platforms because arrays can achieve the necessary breadth, throughput, flexibility, reproducibility, and robustness. See Jenkins et al., Proteomics, 1:13-29 (2001). Improvements in protein separation technologies and selective capture chemistries will accelerate the development of chip and solution arrays.

The technical challenge of high-throughput analysis of the proteome should not be underestimated. Proteomics is more difficult than genomics because proteins have more diverse physicochemical properties and structures that make both their separation, identification, sequencing, and quantification quite challenging. Moreover, unlike nucleic acids, proteins do not hybridize to complementary sequences. In addition, there is no protein equivalent of the polymerase chain reaction. Thus, proteomics requires other means of separating proteins in complex mixtures and identifying both low-and high-abundance species. Although 2D gels are currently the most widely used separation tool in proteomics, it is also worth noting that reverse phase HPLC, capillary electrophoresis, isoelectric focusing and related hybrid techniques also provide means of resolving complex protein mixtures. See Page et al., Proc. Natl Acad. Sci., 96:12589-12594 (1999).

There are a number of critical disadvantages to 2-DE. (i) A well recognized limitation of 2-DE is its inability to reveal mid- to low-abundance proteins. See Figeys et al., Tibtech, 18:483 (2000); Gygi et al., Proc. Natl Acad. Sci., 97:9390-9395 (2000). Unfortunately, many classes of important proteins involved in signal transduction and cellular regulation, such as transcription factors, protein kinases, and phosphatases are present in low copy number and therefore not directly detected on 2-DE. (ii) Comparative proteomics by 2-DE is hampered by variations in the position of the protein spots following separation and this is confounded by additional shifts due to post-translational modifications. (iii) Importantly, 2-DE does not resolve species below 10 kDa and thus cannot report on levels of endogenous peptides such as chemokines and degradation products of larger proteins produced in pathological states. (iv) 2-DE is poorly suited to handling very large or very small sample volumes. (v) Finally, the method is both slow and labor-intensive, typically requiring more than 10 hours per sample.

A broadly applicable approach for protein analysis using an isotope-coded affinity tag (ICAT) has recently been reported. See Gygi et al., Nat. Biotechnol., 17:994-998 (1999), and WO 00/11208, “Rapid Quantitative Analysis of Proteins or Protein Function in Complex Mixtures,” each of which is incorporated herein by reference in its entirety. The reagent consists of biotin for affinity selection, a linker that contains eight light (hydrogen) or heavy (deuterium) isotopes of hydrogen for mass tagging, and a Cys-reactive group (iodoacetamide) to derivatize proteins. Differential labeling involves using two isotopic reagents for two samples in comparative profiling. Samples are mixed following the ICAT derivatization step, proteolyzed together, tagged peptides are affinity purified using Streptavidin, and may be fractionated following extraction from Streptavidin prior to mass spectral analysis. The ratio of mass peak amplitude of peptides from proteins differentially labeled with heavy and light mass tags gives a measure of the relative amounts of each protein. The ICAT method, using a heavy reagent and a light reagent, is limited to differential analysis of two samples.

ICAT has a number of shortcomings. First, ICAT only comes in two masses (light and heavy) that differ by just 8 mass units, but there are applications that require comparisons of more than three or even more states, not just two. Second, cysteine (Cys) is one of the least abundant amino acids. For example, the frequency of arginine is about 5.6% compared to the frequency of cysteine, which is about 2.2%. See FIG. 1. Indeed, about 97% of the sequences contained in the GenBank® database (http://www.ncbi.nlm.nih.gov/) contain arginine while only 84.7% contain cysteine. Thus, more than 15% of such sequences would be outside the scope of a method that targeted cysteine. Furthermore, cysteine is even more underrepresented in proteins/peptides smaller than 10 kDa or 5 kDa (only ˜80% or 57%, respectively, contain Cys) and totally absent in many classes of signaling molecules such as short peptide hormones and neurotransmitters (e.g., dynorphins, enkephalins, substance P, vasoactive intetinal peptide, LHRH, growth hormone-releasing hormone, glucagons-like peptide, bradykinin, angiotensin, etc.). Third, the deuterium mass tags add a number of steps to ICAT synthesis, making the reagent slow and expensive to prepare, prohibitively so in large quantities. Fourth, the iodoacetamide moiety is not the best Cys-reactive moiety. It has a preference for Cys, but can also react with methionine and histidine. See Haugland et al., “Handbook of Fluorescent Probes and Research Chemicals,” 6^{th} Ed., 49-50 (1996). Furthermore, the iodoacetamide reactive group is unstable in light and can result in more than one product with Cys, thus generating heterogeneity and complicating the bioinformatics analysis.

The post-translational modification of proteins is known to be an important mechanism for regulating protein level and activity. Levels of amino acid modification in different systems are important in ascertaining disease states. Thus, the ability to target post-translational modifications provides another reason for detecting and quantitating amino acids that are involved in such processes. The resulting information may have critical importance in ascertaining the presence or risk of developing disease.

Certain amino acids are more commonly involved in post-translational modification than others. Arginine is subject to a number of important post-translational modifications. Similarly, post-translational glycation of proteins is a significant metabolic feature. Protein glycation usually involves condensation of arginine or lysine with dicarbonyl compounds, such as 3-deoxyglucosone, and the end-products have been implicated in a number of diseases processes, including diabetes, renal insufficiency, macrovascular disease, and Alzheimer's disease.

Ideally, methods for protein analysis would be capable of detecting and quantitating levels of post-translational modification, and distinguishing such modified proteins from unmodified proteins.

There is a need for broad spectrum analytic methods and reagents that can target native and post-translational modified proteins. Furthermore, there is a need for such reagents that can be synthesized quickly and inexpensively from commercially available materials.

There is, therefore, a need in the art for methods of quantitating proteins or peptides, including those present only in small quantities. This invention provides methods and reagents to overcome current limitations in traditional analyses performed in proteomics. The approach uses affinity labeled protein reactive reagents that allow for selective isolation of peptide/protein fragments from a complex mixture with or without digestion of the proteins. The present invention provides such a method for detection of extremely small quantities of proteins or peptides, i.e., in the femtomole (10⁻¹⁵ moles) range, and further provides other related advantages.

### SUMMARY OF THE INVENTION

The present invention provides bioanalytical methods and reagents for multiplexed, quantative analysis of proteins. The reagents of the invention react with amino acids or other protein components or structures (i.e., targets) and function as mass tags. The invention typically involves chromatographic separation of the protein/mass tag adducts coupled to mass spectrometric based methods for the quantitative analysis. In certain preferred embodiments, the reagents comprise moieties that permit isolation of proteins from complex mixtures, such as biological fluid or tissue. The reagents may also optionally comprise moieties to adjust the mass, size, or other properties of the reagent.

The reagents of the invention provide for differential labeling of the isolated peptides or the reaction products from enzymatic assays. The mass differentiated reagents can serve as internal standards. As a result, the reagents of the invention facilitate quantitative determination by mass spectrometry of the relative amounts of the proteins in samples. The affinity label serves as a means to obtain selective enrichment and thus may be used to target even proteins that are present in low abundance.

### BRIEF DESCRIPTION OF THE FIGURES

FIG. 1 is a bar chart which shows the relative frequency of various amino acids in the human proteome.

FIG. 2 shows illustrative examples of the PMT reagents of the present invention.

FIG. 3 shows a method for analyzing peptides by MS/MS.

FIG. 4 shows another embodiment of a method for analyzing peptides by MS/MS.

FIG. 5 is a synthetic scheme for synthesizing carboxyl phenyl glyoxal PMT reagents.

FIG. 6 demonstrates examples of an additional family of PMT reagents of the present invention.

FIG. 7 shows the chemical reaction of several PMT reagents that react with thiol groups.

FIG. 8 shows the reaction of a PMT reagent with a phosphoprotein.

FIG. 9 shows the Mass Spectrum of the product of the reaction between PMT Target 1 with Angiotensin II as described in Example 14.

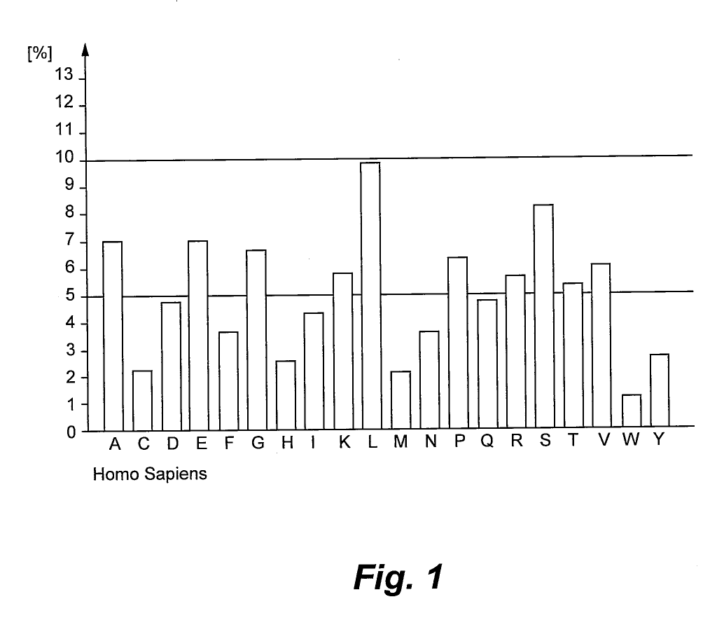

### DETAILED DESCRIPTION OF THE INVENTION

The present invention is directed to novel protein modification reagents, and the manufacture and use of such reagents. These reagents are useful for fractionation and quantitative (differential) profiling of proteins in a complex mixture. The reagents of the present invention are referred to herein as protein mass tag (“PMT”) reagents. The PMT reagents of the invention may be useful as single tagging reagents, or more preferably, as sets of two or more substantially similar but differentiable tagging reagents. See, “Mass Tags for Quantative Analysis of Proteins and Protein Function in Mixtures,” U.S. Provisional Application Ser. No. 60/243,394 (filed Oct. 25, 2000); “Mass Tags for Quantative Analysis of Proteins and Protein Function in Mixtures,” U.S. Provisional Application Ser. No. 60/295,386 (filed May 31, 2000), U.S. Provisional Application Ser. No. 60/296,064 (filed Jun. 5, 2001), and “Strategies for Mass Spectrometry-Based Protein Separation and Analysis Using Mass Tags,” U.S. Provisional Application Ser. No. 60/306,747 (filed Jul. 19, 2001), each of which is incorporated herein by reference in its entirety.

A number of different technologies have been deployed to separate, analyze and identify proteins. Typically, identification by mass spectrometry (MS) involves analysis of isolated proteins or peptide fragments, followed by mapping or tandem MS to obtain sequence information. One strategy that has been used to differentiate the resulting spectra involves tagging the proteins with reagents having different masses (“mass tags”). The use of such mass tags allows a number of different samples to be analyzed at the same time and directly compared.

The protein mass tag (PMT) reagents of the present invention comprise an “amino acid reactive” moiety that is capable of reacting with “protein functional groups” including, but not limited to, an amino acid, modified amino acid, post-translationally modified amino acid, wherein said post-translational modification can occur on an amino acid or a sugar of a glycosolated protein, a set of amino acids, a digested peptide or protein fragment or any other protein structure. The adduct of the PMT reagent and the protein can be analyzed by mass spectrometry, e.g., electrospray ionization (ESI) MS/MS or matrix assisted laser desorption/ionization (MALDI). Proteins originating from different sources can be distinguished based on the mass difference of the PMT reagents. The sequence of the subject proteins can be determined by protein mapping or by tandem mass spectrometry (MSⁿ).

The PMT comprises, at least, an amino acid reactive moiety. It may also comprise one or more accessory moieties and/or one or more recognition moieties. The portion of the PMT that contains mass difference, from one PMT to the next, may be found in one or any combination of the amino acid reactive moiety, the accessory moiety or the recognition moiety.

The “accessory moiety” or moieties (AM) (which are comprised by the PMT reagents in some embodiments) can be used to adjust the mass, size, or other physical property of the PMT reagent. In some preferred embodiments, the PMT reagent comprises a “recognition group” to aid in the isolation of the labeled protein.

The present invention is directed to novel PMT reagents and their use in protein isolation and identification. In its simplest form, the PMT reagents of the present invention comprise a protein reactive moiety (alternatively referred to here as an amino acid reactive moiety). The protein reactive moiety is a chemical functionality that reacts specifically with protein or peptide components. The protein reactive moiety will typically, but need not necessarily, form a covalent bond between the PMT reagent and the protein or peptide functionality for which it binds specifically. The protein reactive moiety may bind a specific amino acid side chain (e.g., the thio group of cysteine; the guandinium group of arginine; the imidazolium group of histidine) or a post-transitionally modified amino acid side chain. Alternatively, the protein reactive moiety may have an affinity for certain three-dimensional structural elements of proteins or peptides, or to defined amino acid patterns or any other element of a protein or peptide that could be chemically reactive.

In the preferred embodiments of the present invention, it is desirable to use PMT reagents that will react with most proteins or peptides in a sample, but will not react with, or subsequently tag, each protein or peptide more than once or twice. With multiple tagging, the interpretation of resultant MS analysis can become too difficult to provide meaningful data. As described above, the ICAT reagents target cysteine amino acid side chains, which occur at a relative frequency of 2.2%. In the preferred embodiments of the present invention, the PMT reagents are designed to react with the side chain of arginine, which occur with a relative frequency of 5.6%. This greater frequency is particularly important when tagging proteins from a sample that have been or will be cleaved into peptide fragments to facilitate analysis.

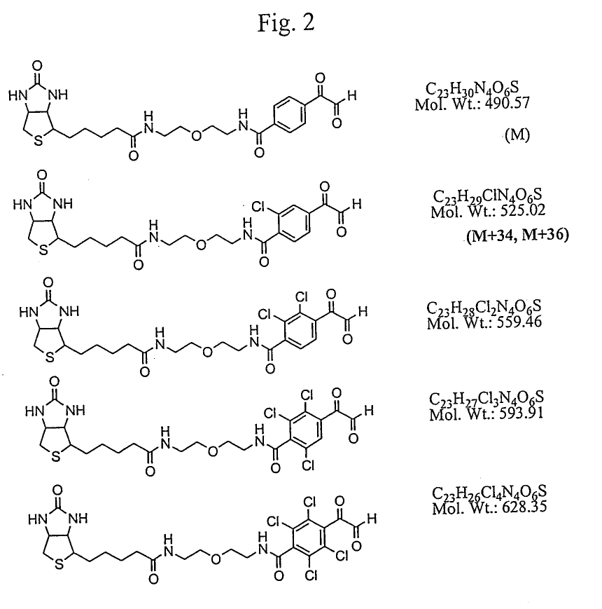

The second defining feature of the PMT reagents of the present invention is the ability to serve as a mass tag. It is desirable for the PMT reagents of the present invention to have chemical variability that will allow the creation of a “family” of PMT reagents. While each member of such family falls within the scope of the present invention, it is the ability to be a member of a family of PMT reagents, comprising a plurality of members, which is essential for a reagent to serve as a mass tag. Several examples are presented below that represent different families of PMT reagents of the present invention. In FIG. 2, for example, the family members all have the same chemical backbone structure. The only difference between the members of the family is the extent of halogenation of the phenyl ring. The compounds synthesized in Examples 12 and 13, below, also represent members of a common family of PMT reagents. The difference between the two members, in this case, being the presence of an ethoxy group versus a methoxy group on the phenyl ring. In both of these cases, the differences between the family members changes the mass of the PMT reagent and—when attached to the tagged protein or peptide—will be able to provide a way of differentiating the various family members by mass. It is important to assure that the variations that occur between members of a family of PMT reagents that allow for mass differentiation do not substantially affect the ability to react, or rate of reaction, between the protein reactive moiety and the protein or peptide.

In preferred embodiments of the present invention, the PMT reagents also comprise a recognition moiety. The recognition moiety is a chemical or biochemical functionality that forms a specific binding pair—covalent or noncovalent—with another chemical or biochemical functionality. Nonlimiting examples of recognition groups useful in the present invention include biotin and short nucleic acid sequences, preferably having between 5 and 50 bases. Further examples are presented below.

In addition, the PMT reagent of the present invention may also include one or more accessory moieties. Such accessory moieties can serve any particular function that may be required or advantageous in using the PMT reagents of the present invention for a particular application. For example the accessory moiety may be a fluorescent chemical functionality. Such functionality would allow identification of tagged species in a sample. An accessory group may also be employed that allows for or enhances separation of the proteins or peptides tagged by the PMT reagents. The accessory moiety may also be the portion of the PMT reagent used as the mass tag.

Although many PMT reagents fall within the scope of the present invention, some specific examples given herein can be represented as follows:

RM-PRM

wherein RM is the recognition moiety and PRM is the protein/amino acid reactive moiety. In preferred embodiments, the PMT reagents of the present invention RM is biotin and PRM reacts specifically with the side chain of arginine amino acid residues. In this representation, RM and PRM may be joined by a linker, L, or may be directly attached to each other. In some cases the RM and PRM may be the same chemical moiety. The mass tag portion of the PMT reagents (the area where chemical derivatization occurs to yield different masses for the different family members) may occur on the RM, the PRM, the linker (L) or on an accessory moiety (AM).

Specific families of PMT reagents have the following structures.

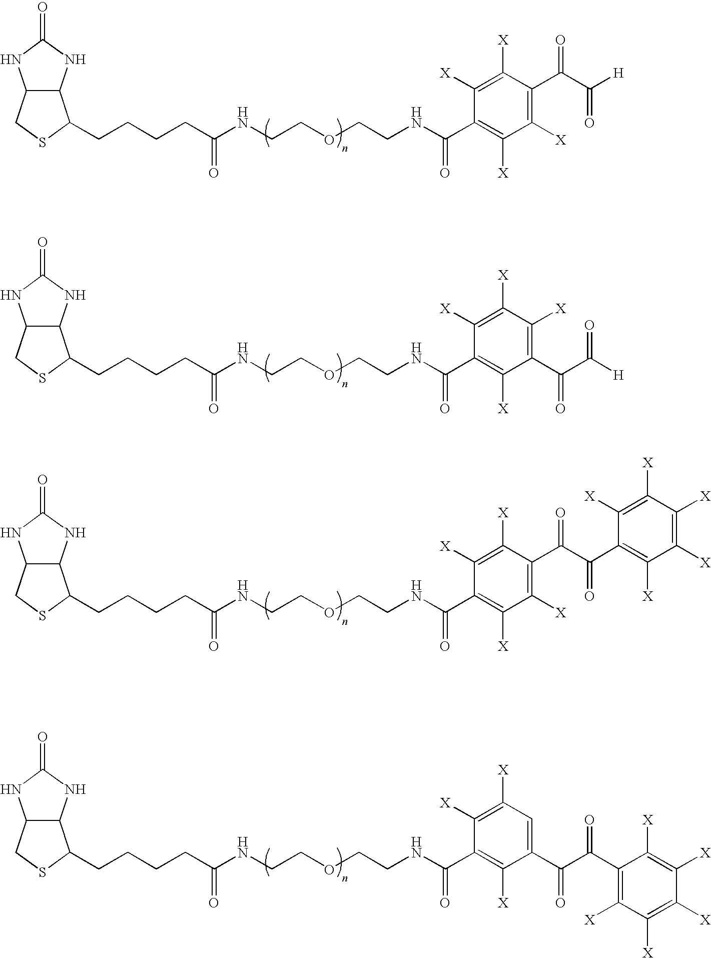

wherein
X is independently selected from the group consisting of H, D, OH, OD, R, OR, OSiR₃, Cl, Br, I, F, SH, SR, NH₂, NHR, and NR₂;
R is selected from the group consisting of an optionally substituted: C₁-C₂₀ alkyl, C₂-C₂₀ alkenyl, C₂-C₂₀ alkynyl, including deuterium substitutions; and
n=0-10.

The term “protein mass tags” as used herein, generally refers to a chemical moiety that is used to uniquely identify a protein or peptide in a sample.

A tag which is preferred for use in an assay according to the present invention possesses several attributes:

1) It is capable of being distinguished from the other tags used in the assay. This discrimination from other tags is based on the mass of the tag.

2) The tag is capable of being detected when present at 10⁻⁶ to 10⁻²² mole.

3) The tag possesses a chemical moiety that allows it to become attached to the protein or peptide that the tag is intended to uniquely identify.

4) The tag is chemically stable toward the manipulations to which it is subjected, including attachment and any manipulations of the sample while the tag is present.

5) The tag does not significantly interfere with the manipulations performed on the sample while the tag is present.

The PMT reagents of the present invention have broad use in proteomics. Although the targets may be referred to herein as “proteins,” the scope of the invention includes protein fragments, peptides, the products of enzymatic reactions, as well as other amino acid containing molecules (e.g., glycoproteins and post-translationally modified proteins).

The mass difference between members of a PMT reagent family is typically due to substitutions with related chemical moieties. For example, the reagents may be modified with one or more halogens. Single substitutions of F, Cl, I and Br would yield a set of five different forms or “versions” of the PMT reagent, each having a different mass, from the “heaviest” (the iodine substituted reagent, PMT-I) to the “lightest” (the non-substituted reagent, PMT-H). The use of any two versions of a PMT reagent would be sufficient to distinguish tagged protein from two samples (e.g., normal and diseased).

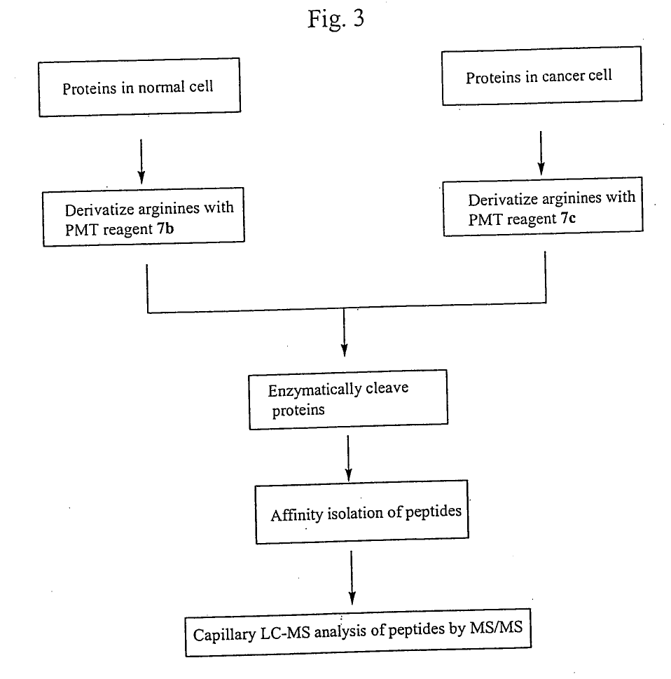

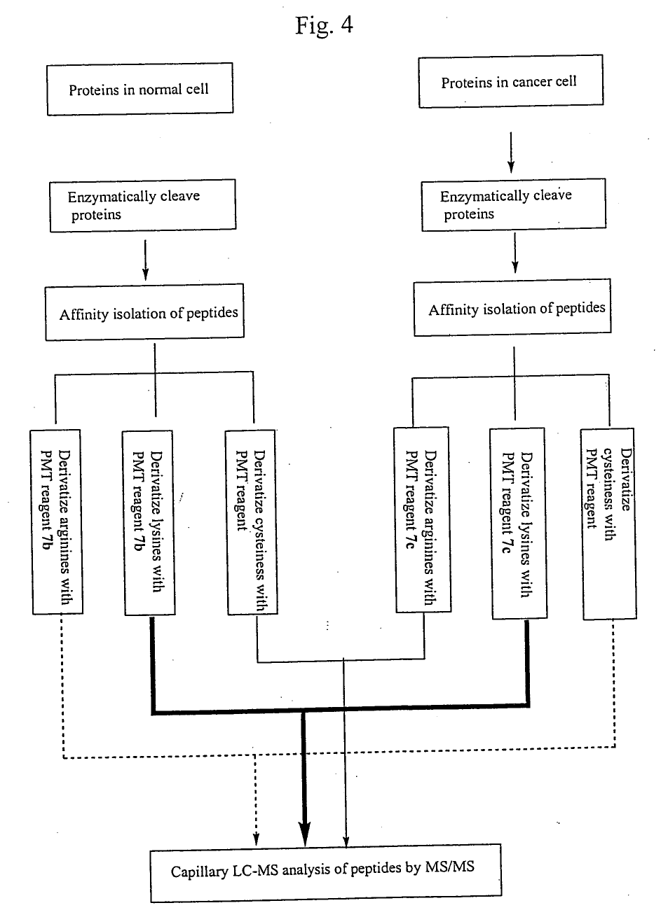

Because they have different masses, the PMT reagents (and therefore their protein adducts) are distinguishable by mass spectrometry. As an illustrative example, two versions of a PMT reagent, identical except for the mass tag they carry, may be used. One version of the PMT reagent (PMT-F) is contacted with a first sample while the other version (PMT-Cl) is contacted with a second sample. Once isolated, the labeled proteins from the two samples are simultaneously analyzed by mass spectrometry. Peaks corresponding to proteins from the first sample can be differentiated from peaks corresponding to proteins from the second sample based on mass: the peaks separated by the difference in mass between PMT-F and PMT-Cl. This process allows for multiplexing of analysis by analyzing two or more samples at the same time. In addition, provided the samples have been handled in the same way, the differentially labeled proteins serve as internal standards, facilitating quantitative determination by mass spectrometry of the relative amounts of the proteins in the different samples. Variations on this basic method are shown in FIGS. 3 and 4.

After analysis by mass spectrometry, the ratio of the ion intensities for a labeled pair of peptide fragments provides the relative abundance of the parent protein in the original populations. In addition, through techniques well known in the art, the peptides may be further analyzed to determine their sequence. For example, tandem mass spectrometry MS/MS may be performed on these peptides, followed by database searches to match fragmentation patterns and identify the peptide in question.

Using a plurality of distinguishable versions of a PMT reagent allows the simultaneous analysis of additional samples. For example, the use of the five versions of the halogen-substituted PMT reagent described above would allow a control sample to be directly compared to four experimental samples at the same time. Thus, the PMT reagents of the present invention provide a powerful tool for rapidly quantitatively analyzing protein expression and can function as a complementary method to study gene expression and perturbation induced changes.

In certain preferred embodiments, the PMT reagents of the present invention react specifically with arginine amino acid residues. Because of the specificity of the reagents for particular protein structures (e.g., amino acid side chain), the method can be used to distinguish between functionally different but isobaric species. For example, the post-translational modification of arginine to a modified form may be difficult to pick up by routine mass spectrometry. However, if the post-translational modification removed or significantly altered the guanidine group, certain arginine reactive moieties of the invention would preferably react with arginine and not the post-translationally modified form. The relative amounts of such species could be determined by selectively targeting the native and post-translationally modified amino acids with different PMTs.

Throughout the invention, reference is made to samples containing proteins and/or peptides. Typically, such samples are biological samples such as blood or serum. However, such biological samples include not only samples obtained from living organisms (e.g., mammals, fish, bacteria, parasites, viruses, fungi and the like) or from the environment (e.g., air, water or solid samples), but biological materials which may be artificially or synthetically produced (e.g., phage libraries, organic molecule libraries, pools of genomic clones and the like). Representative examples of biological samples include biological fluids (e.g., blood, semen, cerebral spinal fluid, urine), biological cells (e.g., stem cells, B or T cells, liver cells, fibroblasts and the like), and biological tissues.

In certain embodiments of the present invention, the proteins may be first isolated from the sample before they are then labeled with a PMT reagent and analyzed by mass spectrometry.

In certain embodiments of the present invention, it is advantageous to separate the proteins in a sample into fractions before tagging and detection. This can be accomplished by a wide variety of methods familiar to those skilled in the art. The separation or fractionation of proteins or peptides may be accomplished by a variety of techniques, including 2-DE, capillary electrophoresis, micro-channel electrophoresis, HPLC, size exclusion chromatography, filtration, polyacrylamide gel electrophoresis, liquid chromatography, reverse size exclusion chromatography, ion-exchange chromatography, reverse phase liquid chromatography, pulsed-field electrophoresis, field-inversion electrophoresis, dialysis, and fluorescence-activated liquid droplet sorting. Alternatively, the proteins or peptides may be bound to a solid support (e.g., hollow fibers (Amicon Corporation, Danvers, Mass.), beads (Polysciences, Warrington, Pa.), magnetic beads (Robbin Scientific, Mountain View, Calif.), plates, dishes and flasks (Corning Glass Works, Corning, N.Y.), meshes (Becton Dickinson, Mountain View, Calif.), screens and solid fibers (see Edelman et al., U.S. Pat. No. 3,843,324; see also Kuroda et al., U.S. Pat. No. 4,416,777), membranes (Millipore Corp., Bedford, Mass.), and dipsticks. If the proteins or peptides are bound to a solid support, within certain embodiments of the invention the methods disclosed herein may further comprise the step of washing the solid support.

In some embodiments it may be desirable to cleave or “digest” the proteins in a sample, either before or after tagging. This can be accomplished by a wide variety of methods familiar to those skilled in the art. For example, the proteins in the sample may be digested with cyanogen bromide (CNBr) or enzymatically digested (e.g., with trypsin) either before or after being labeled.

A wide range of mass spectrometric techniques also may be useful in the present invention. Representative examples of suitable spectrometric techniques include time-of-flight (TOF) mass spectrometry, quadrupole mass spectrometry, magnetic sector mass spectrometry and electric sector mass spectrometry. Specific embodiments of such techniques include ion-trap mass spectrometry, electrospray ionization (ESI) mass spectrometry, ion-spray mass spectrometry, liquid ionization mass spectrometry, atmospheric pressure ionization mass spectrometry, electron ionization mass spectrometry, fast atom bombard ionization mass spectrometry, MALDI mass spectrometry, photo-ionization time-of-flight mass spectrometry, laser droplet mass spectrometry, MALDI-TOF mass spectrometry, APCI mass spectrometry, nano-spray mass spectrometry, nebulised spray ionization mass spectrometry, chemical ionization mass spectrometry, resonance ionization mass spectrometry, secondary ionization mass spectrometry and thermospray mass spectrometry.

By labeling the proteins with a PMT reagent that comprises a recognition moiety (e.g., biotin), the PMT reagents also serve as a means to obtain selective enrichment of proteins. Use of a recognition moiety is particularly useful when the methods of the invention are applied to proteins that are present in small amounts or when the proteins exist in a complex mixture. In these situations, the recognition moiety can function as a “handle” to allow isolation and concentration of the labeled protein.

The recognition moiety can be any moiety that has an affinity for another species. The list of possible recognition moieties could be expanded to hundreds or thousands of different chemistries, encompassing specific capture agents such as oligonucelotides and/or antibodies as well as ligands for particular receptors, cofactors for proteins, and so forth. It will be appreciated by those skilled in the art that pairs of interacting molecules can be exploited in two ways: (1) with a stationary phase to capture a “ligand” and (2) with a stationary phase to capture a counterligand “receptor.” A list of some but not all types of such pairs in biological systems is listed in Table I.

TABLE I

| LIGAND | COUNTERLIGAND/RECEPTOR |
|---|---|
| Cofactors | Enzymes |
| Lectins | Polysaccharides, glycoproteins |
| Nucleic acid | Nucleic Acid binding protein (enzyme or histone) |
| Biomimetic dyes | Kinases, phosphatases, Dehydrogenases etc. |
| Protein A, Protein G | Immunoglobulins |
| Metals ions | Most proteins can form complexes with metal ions |
| Enzymes | Substrate, substrate analogues, inhibitor, cofactors |
| Phage displays | Proteins, peptides, any type of protein |
| DNA libraries | Complementary DNA |
| Aptamers | Proteins, peptides, any type of protein |
| Antibody libraries | Any type of protein |
| Carbohydrates | Lectins |
| ATP | Kinases |
| NAD | Dehydrogenases |
| Benzamide | Serine Protease |
| Phenylboronic acid | Glycoproteins |
| Heparin | Coagulation proteins and other plasma proteins |
| Receptor | Ligand |
| Antibody | Virus |

It should be understood that countless other examples of specific interactions are known and can be exploited. In this way, for example, the PMT-labeled proteins may be isolated by a streptavidin affinity chromatography and then analyzed by LC/MS. In a simple example, the recognition moiety could be biotin, and the affinity column counterligand could be streptavidin. In another embodiment, the recognition moiety could be a nucleic acid, which could be isolated by hybridization with its complementary sequence.

In certain preferred embodiments, the amino acid reactive moiety of the PMT reagent is a 1,2 dicarbonyl moiety, making the PMT reagent specific for the amino acid residue, arginine. The 1,2 dicarbonyl moiety condenses with the guanidino moiety of arginine to yield an imidazolone adduct. In other preferred embodiments, the amino-acid reactive portion of the reagent binds to other amino acid residues (either one or more than one) or other protein structural elements, such as disulfide bonds.

In one series of preferred embodiments, the PMT reagents comprise biotinylated phenylglyoxals. The dicarbonyl structures in these reagents provide the chemistry for condensation with the guanidine moiety of the arginine side chain. The biotin allows the tagged peptides to be readily separated from the mixture, for example by using a chromatography column. Using a number of different versions of a PMT reagent, each having different masses (e.g., created by different halogen substitutions), allows the protein adducts to be distinguished by mass spectrometry. PMT reagents comprising biotinylated phenyl glyoxals can be synthesized from commercially available materials and thus offer rapid and inexpensive access to a diverse set of reagents.

As discussed above, PMT reagents that react with arginine provide broad coverage of the proteome because arginine occurs in proteins with a high relative frequency. Furthermore, because lysine residues can be converted to arginine, these same PMT reagents can also be applied to proteins that contain lysine. The lysine may be derivatized by first converting the ε-amino group to a guanidine with O-methyl isourea to yield homoarginine. The resultant guanidine group is then condensed, as discussed above, with the phenyl glyoxal moiety of the PMT reagent. The chemistry of this modification has been developed to selectively derivatize lysine to homoarginine without the concomitant conversion of the amino-terminus of the peptides. This technique allows assessment of the total arginine and lysine in protein mixtures. Significantly, it also allows the ratio of lysine/arginine to be determined.

An example of a family of PMT reagents comprising biotin and a phenylglyoxal moiety is shown in FIG. 2. Alkyl and aryl glyoxals are dicarbonyl compounds that can modify arginyl residues in proteins. The use of substituted phenylglyoxals serves a twofold purpose. First, the dicarbonyl moiety reacts specifically with arginine residues. Second, the phenyl portion provides the basis for substitution with different atoms and allows the reagent to act as a mass tag, i.e., allows the various versions of PMT reagents to be differentiated from one another when analyzed by mass spectrometry. As shown in the examples of FIG. 2, the basic PMT structure remains the same—a biotin residue attached via a organic chain to the phenylgyloxal moiety. The difference between the molecules is the extent of chlorination of the phenyl group. The five species of this family of PMT reagents should all exhibit relatively the same ability to react with arginine residues, the same ability to be captured by streptavidin, and the same chromatographic properties.

While in this example the extent of chlorination of the phenyl ring is used to create the mass difference between the different family members, other chemical substituents could be used alternatively. For example, additional chemical substitutions could be made on the accessory moiety. Alternatively, the phenyl group could be methoxylated or fluorinated rather than chlorinated. The number of possibilities of creating variations on this basic structure is large, given the number of available positions for substitution and the number of possible chemical substituents. However, in preferred embodiments, the type and location of substituents is limited for any given experiment or assay. In order to obtain relative quantitative information regarding the composition of different samples, it is essential that the chemical reactivity of the PMT reagent with the reactive portion of the relevant proteins or peptides be essentially the same.

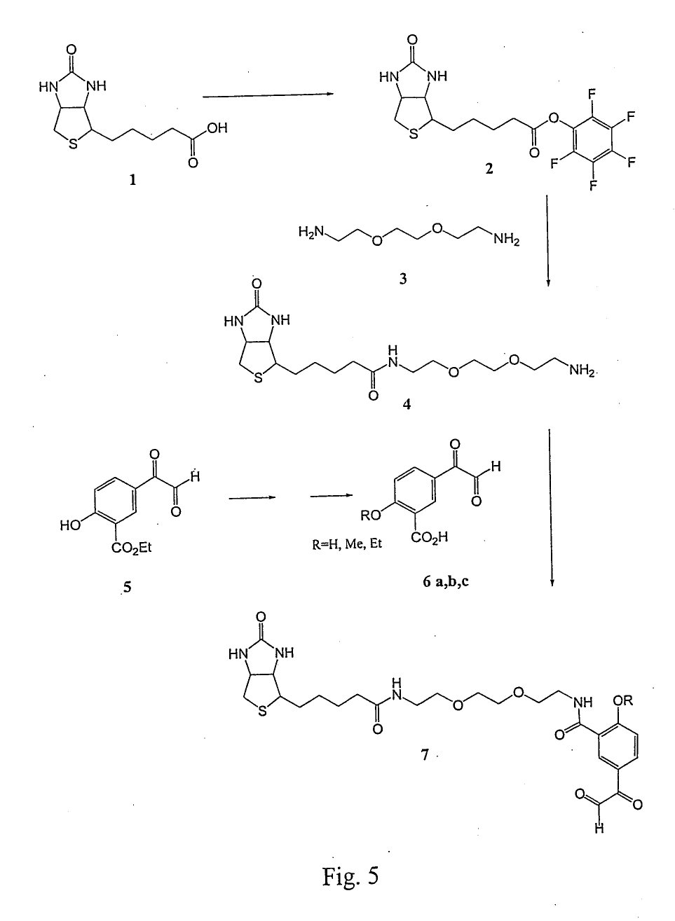

The PMT reagent of the present invention can also comprise carboxyl phenylglyoxals (or other substituted di-ketones). A synthetic process for making the unsubstituted carboxyl phenylglyoxals is shown in FIG. 5. These dicarbonyl structures not only provide the chemistry for condensation with the guanidine moiety of arginine side chain but also carry mass tags that allow them to be distinguished by mass spectrometry. Tri-substituted benzenoid derivatives carrying four functionalities, while more difficult to synthesize de novo, are available commercially and can be readily incorporated into PMT reagents. A phenyl glyoxal derived from 3-carboethoxy 4-hydroxy phenylglyoxal, which is commercially available material, shown in FIG. 5 (structure 5).

Referring to FIG. 5, the hydroxyl group in structure 5 is first alkylated to yield the intermediate alkoxy phenyl glyoxal, the latter being subsequently hydrolyzed to yield the alkoxy substituted carboxy phenyl glyoxals (—OMe and —OEt functioning as the mass tags). A biotin amine can be attached at the carboxyl group to yield the final target. This approach has the advantage of being “modular.” That is, the biotin amine serves as the common intermediate to link the different phenyl glyoxals or other amino acid reactive moieties.

Commercially available biotin is converted to its active ester form and reacted with ethylene dioxy 1,6 amino octane. The resulting amino-linked biotin is purified by chromatography and coupled to appropriately substituted carboxy phenyl glyoxals to yield PMT reagents of the present invention.

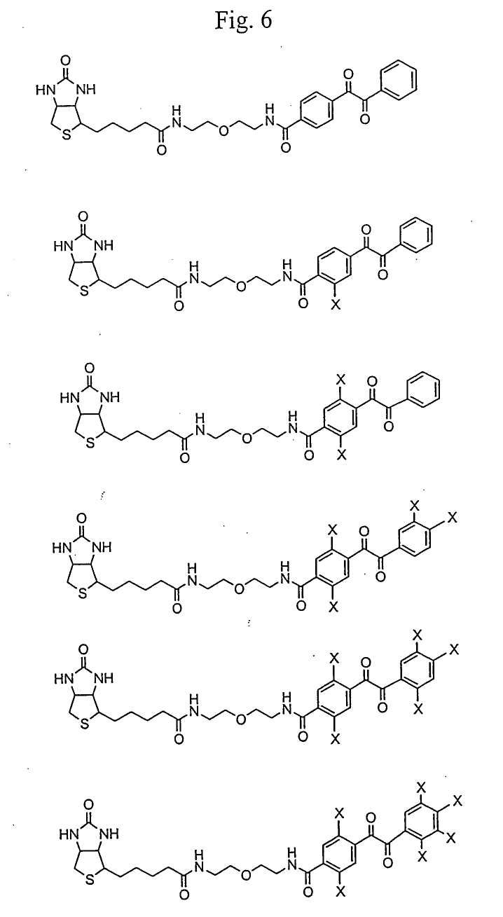

FIG. 6 shows another family of PMT reagents of the present invention. Similar to the compounds shown in FIGS. 2 and 5, these compounds are biotinylated diphenyl diketone moieties. The presence of the second phenyl group provides for more potential diversity in structurally related compounds having differentiated masses.

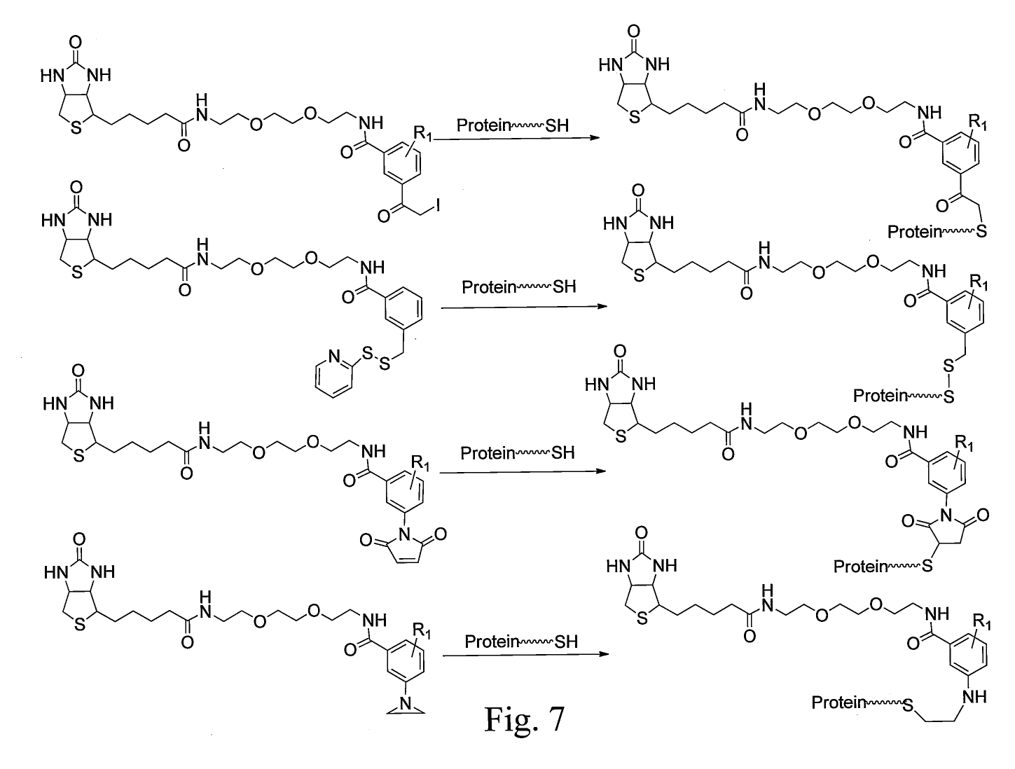

Certain PMT reagents have amino acid reactive moieties that are thiol reactive moieties. Their reaction with cysteine residues yield mass tagged products capable of being affinity purified and/or concentrated for mass spectrometric analysis. The reaction of a number of such PMT reagents (comprising biotin moieties) are shown below in FIG. 7.

The accessory moiety of the PMT reagents can be used for a number of purposes. For example, accessory moieties may be used to increase the mass of the reagent. In addition, accessory moieties can aid in differential binding to peptides (e.g., steric relationships, peptide tertiary or quaternary structure) or aid in separation (e.g., size exclusion, gel separation). Using a fluorescent group as an accessory moiety, as shown below, allows absolute quantitation.

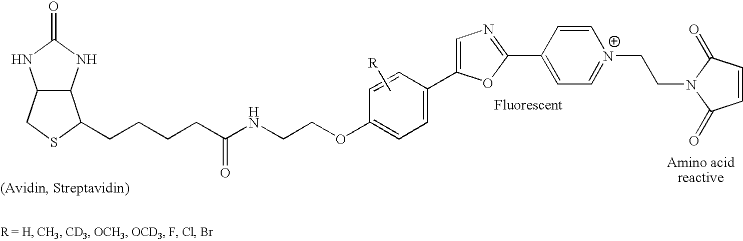

By using such a reagent, the relative quantitation (e.g., the ratio of peak intensities from the two samples) obtained by mass spectrometry may be deconvoluted to obtain absolute quantitation of the tagged proteins from different samples.

As described above, the PMT reagents of the present invention comprise an amino acid reactive moiety, and can be differentiated on the basis of their mass. In addition, a PMT reagent may contain a recognition moiety and/or one or more accessory moiety. In certain embodiments, the same moieties or portions of the PMT reagent may serve more than one of these functions.

In the example shown below, the protein reactive group is fluorescent and also comprises mass tags. In addition to being thiol reactive, the bromobimane moiety is fluorescent. The bromobimane moiety can also be substituted (e.g., R═CH₃, CD₃, C₂H₅, C₂D₅, C₆H₅, C₆D₅ etc.). Thus, the bromobimane moiety can be the portion of the PMT reagent that is substituted in order to provide mass differentiation. Bromobimane derivatives are commercially available from Molecular probes (Eugene, Oreg.).

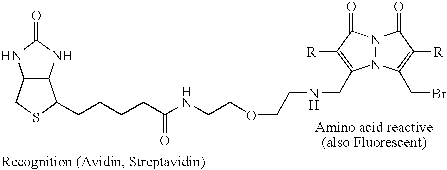

The combination of elements in the PMT reagents of the present invention can be accomplished by a large number of possibilities. For example, the recognition moiety (bipyridyl or phenanthroline with metal binding capacity) could be juxtaposed so that the mass tags (R═H, CH₃, CD₃, C₂H₅, C₂D₅) are remote from the protein reactive group (phenyl glyoxal), as shown below.

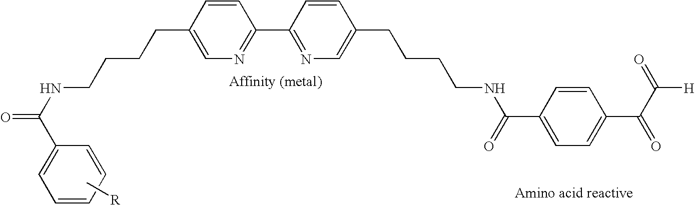

Alternative binding pairs that could be used for affinity purification are nucleic acid duplexes and antigen-antibody interactions. The nucleic acid could also serve as a mass tag with modified bases that do not interfere with the Watson-Crick base pairing. The protein reactive group and the mass tags could also be incorporated at different ends (3′ and 5′ modification at the terminus).

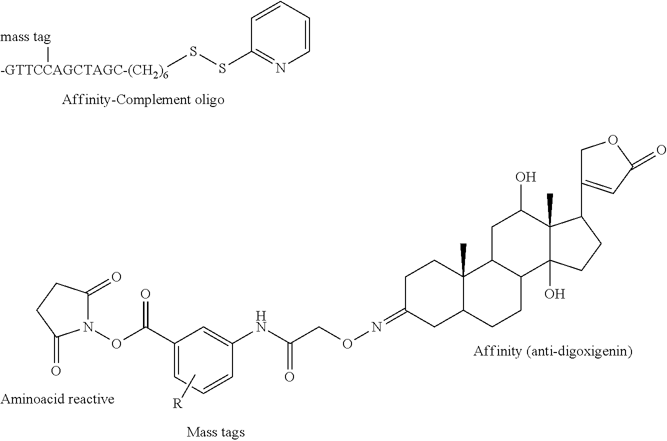

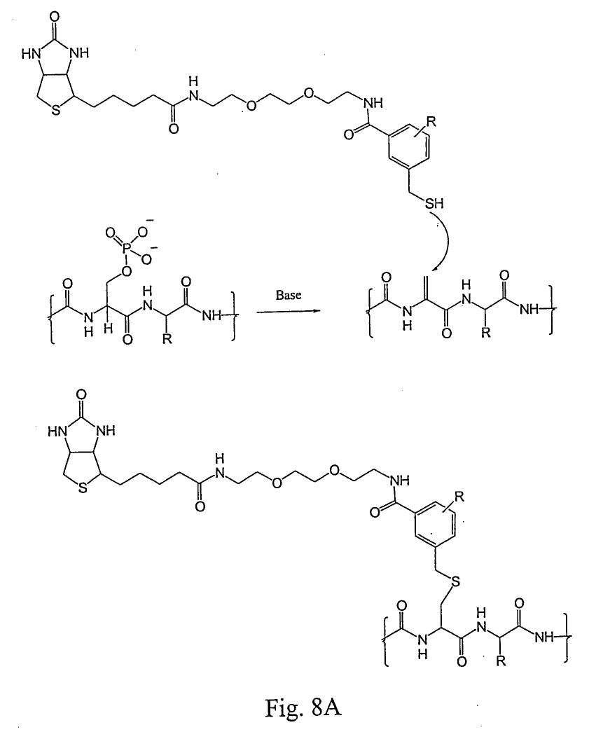

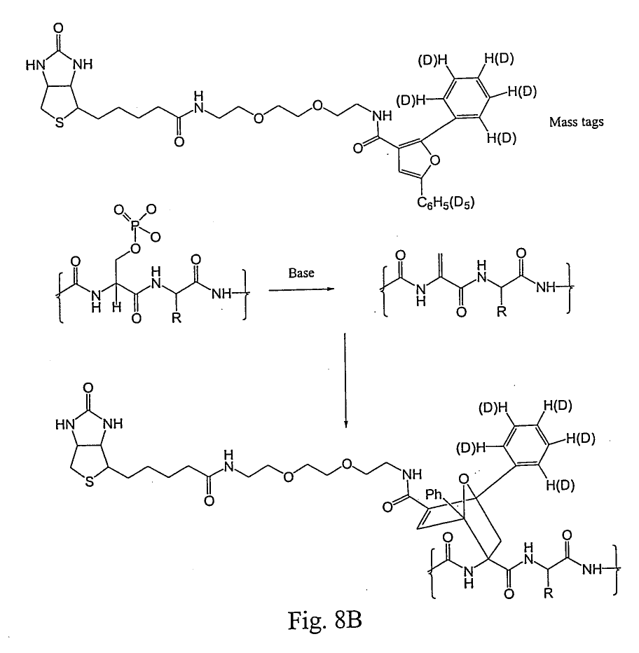

An important post-translational modification of proteins is phosphorylation, which occurs predominantly at the OH of serine, threonine and tyrosine residues. PMT reagents may be used according to the methods of the present invention to isolate and quantitate the extent of phosphorylation. The reagents of this invention can be used to capture phosphoproteins (e.g., serine and threonine only) and determine their relative quantities in two or more samples. See FIG. 8.

In one example, the PMT reagents of the present invention can be applied to perform relative quantification of analytes in two samples using cLC-MS/MS and MALDI MS. First, PMT reagents are prepared that are arginine specific, each with a biotin recognition group. These reagents may then be used to test serum samples to address dynamic range and relative quantification by a number of approaches. For example, proteins in serum can be condensed with PMT reagents and then digested. Alternatively, serum proteins can be digested and then condensed with the PMT reagents. The labeled proteins can then be run through a streptavidin column. LC-MS, MALDI or ESI, can be used to analyze the released biotinylated protein adducts with the PMT reagent.

The following specific examples are provided to better assist the reader in the various aspects of practicing the present invention. As these specific examples are merely illustrative, nothing in the following descriptions should be construed as limiting the invention in any way.

### Examples

### Example 1

### Synthesis of 3-Carbomethoxy-4-hydroxy acetophenone

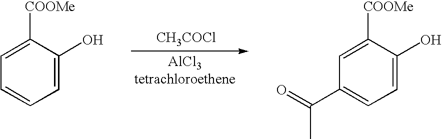

Methyl salicylate (75 g, 0.49 mol) was added to tetrachloroethene (600 mL) followed by acetyl chloride (38.7 g, 0.49 mol). After cooling the reaction mixture to 0° C., anhydrous aluminium chloride (131 g, 0.99 mol) was added over a period of 30 min. and stirred for 4 h. It was further stirred for 4 h at 40-50° C. The reaction was quenched by pouring into ice-cold water. The organic layer was washed successively with water (100 mL), aqueous sodium bicarbonate solution (2×100 mL), water (100 mL) and brine (100 mL). The solvent was evaporated and unreacted methyl salicylate was distilled out using high vacuum. The crude product was recrystallised from pet. ether.

Yield: 40 g (43%).

¹H NMR (CDCl₃) δ 11.2 (s, 1H), 8.5 (s, 1H), 8.0 (d, 1H), 7.0 (d, 1H), 4.0 (s, 3H), 2.5 (s, 3H). See Jen, T., Frazee, J. S., Kaiser, C., J. Med. Chem., 1977, Vol. 20, no. 8, 1029-1035.

### Example 2

### Synthesis of 3-Carbomethoxy-4-ethoxy acetophenone

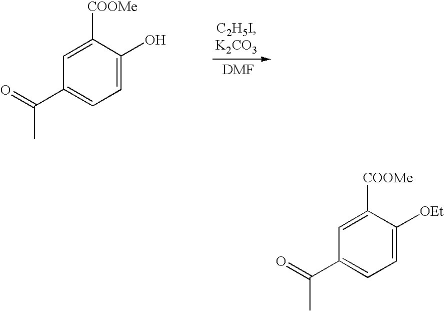

3-Carbomethoxy-4-hydroxy acetophenone (20 g, 0.103 mol) was dissolved in dry DMF (100 mL) and potassium carbonate (15.6 g, 0.113 mol) was added followed by ethyl iodide (19.3 g, 0.123 mol). The reaction mixture was refluxed at about 60° C. for 12 hours. On completion of the reaction (by TLC) the mixture was diluted with water and extracted with ethyl acetate (2×250 mL). The combined ethyl acetate extract was washed with water (250 mL) and brine (250 mL). It was dried over sodium sulfate and concentrated to give the product.

Yield: 22 g (96%).

### Example 3

### Synthesis of 3-Carboxy-4-ethoxy acetophenone

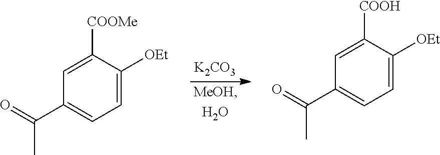

3-Carbomethoxy-4-ethoxy acetophenone (20 g, 0.09 mol) was dissolved in methanol (100 mL). Potassium carbonate (50 g, 0.36 mol) was dissolved in water (100 mL) and added to the above solution. The reaction mixture was stirred at about 60° C. for 7 h. On completion of the reaction (by TLC), the reaction mixture was acidified with 6 N HCl (pH: 2.0) and extracted with ethyl acetate (3×200 mL). The combined extract was washed with water and brine and concentrated to yield the product.

Yield: 17 g ( 91%).

¹H NMR (CDCl₃) δ 11.0 (br, s, 1H), 8.75 (s, 1H), 8.25 (d, 1H), 7.1 (d, 1H), 4.5(q, 2H), 2.6 (s, 3H), 1.6 (t, 3H).

### Example 4

### Synthesis of 3-Carboxy-4-ethoxy acetophenone pentafluorophenyl ester

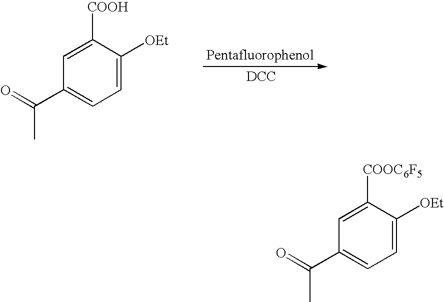

To a solution of 3-carboxy-4-ethoxy acetophenone (17 g, 0.082 mol) in dry 1,4-dioxane (400 mL), pentafluorophenol (18 g, 0.01 mol) was added. The mixture was cooled to about 5° C. and DCC (24 g, 0.116 mol) was added. The reaction mixture was stirred overnight at RT. It was filtered and the solvent evaporated under vacuum. The crude product was purified by column chromatography (silica gel, 60-120 mesh; eluent, pet. ether:ethyl acetate, 5:5).

Yield: 23 g (75%).

¹H NMR (CDCl₃) δ 8.75 (s, 1H), 8.25 (d, 1H), 7.1 (d, 1H), 4.25 (q, 2H), 2.6 (s, 3H), 1.5 (t, 3H).

### Example 5

### Synthesis of 3-Carboxy-4-ethoxy phenylglyoxal pentafluorophenyl ester

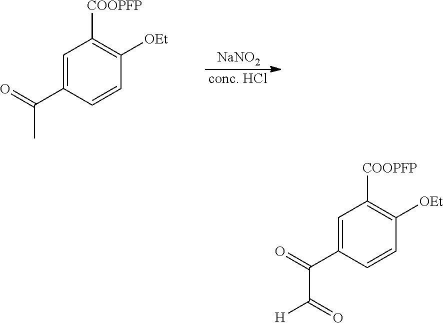

A suspension of 3-carboxy-4-ethoxy acetophenone pentafluorophenyl ester (8 g, 0.021 mol) in a mixture of conc. HCl (7.2 mL) and 1,4-dioxane (21.6 mL) was heated to about 60° C. A solution of sodium nitrite (3.2 g, 0.047 mol) in water (9.4 mL) was added dropwise over a period of 4 h. The reaction was cooled and diluted with water (200 mL). The mixture was extracted with ethyl acetate (3×100 mL) and the combined extract was washed with water and brine. It was then dried (sodium sulfate) and concentrated. The crude product was triturated with ether and filtered.

Yield: 3 g (37%).

¹H NMR (DMSO-d₆) δ 8.8 (s, 1H), 8.4 (d, 1H), 7.4 (d, 1H), 5.9 (d, 1H), 4.2 (q, 2H), 1.4 (t, 3H). See WO 93/17989, “Preparation of Substituted or Unsubstituted Phenylglyoxals.”

### Example 6

### Synthesis of 3-Carbomethoxy-4-methoxy acetophenone

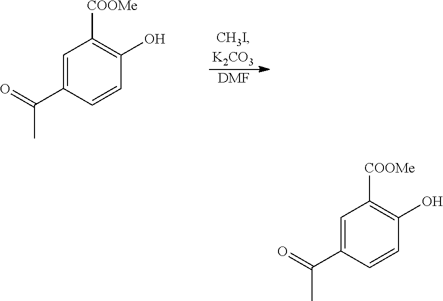

3-carbomethoxy-4-hydroxy acetophenone (20 g, 0.103 mol) was dissolved in dry DMF (100 mL) and potassium carbonate (15.6 g, 0.113 mol) was added followed by methyl iodide (43.7 g, 0.3 mol). The reaction mixture was stirred at RT overnight. On completion of the reaction (by TLC) it was diluted with water and extracted with ethyl acetate (2×250 mL). The combined ehtyl acetate extract was washed with water (250 mL) and brine (250 mL). It was dried over sodium sulfate and concentrated to give the product.

Yield: 20 g (93%).

### Example 7

### Synthesis of 3-Carboxy-4-methoxy acetophenone

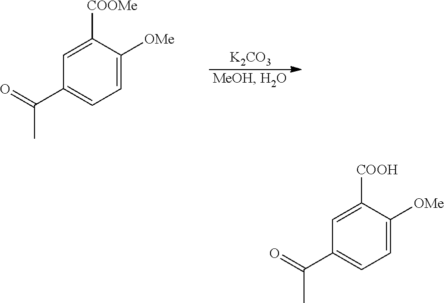

3-Carbomethoxy-4-methoxy acetophenone (20 g, 0.096 mol) was dissolved in methanol (150 mL). Potassium carbonate (61 g, 0.44 mol) was dissolved in water (150 mL) and added to the above solution. The reaction mixture was stirred at about 60° C. for 4 h. On completion of the reaction (by TLC), the reaction mixture was acidified with 6 N HCl (pH: 2.0) and extracted with ethyl acetate (3×200 mL). The combined extract was washed with water and brine and concentrated to yield the product.

Yield: 17.5 g ( 93%).

¹H NMR (CDCl₃) δ 10.5-11.0 (br, s, 1H), 8.75 (s, 1H), 8.25 (d, 1H), 7.1 (d, 1H), 4.1(s, 3H), 2.6 (s, 3H).

### Example 8

### Synthesis of 3-Carboxy-4-methoxy acetophenone pentafluorophenyl ester

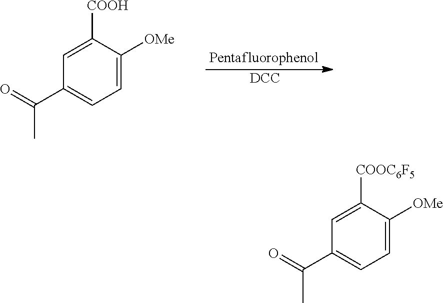

To a solution of 3-carboxy-4-methoxy acetophenone (17 g, 0.088 mol) in dry 1,4-dioxane (350 mL), pentafluorophenol (17.7 g, 0.096 mol) was added. The mixture was cooled to about 5° C. and DCC (23.5 g, 0.113 mol) was added. The reaction mixture was stirred overnight at RT. It was filtered and the solvents were evaporated under vacuum. The crude product was purified by column chromatography (silica gel, 60-120 mesh; eluent, pet. ether:ethyl acetate, 5:5).

Yield: 19 g (60%).

¹H NMR (CDCl₃) δ 8.75 (s, 1H), 8.25 (d, 1H), 7.1 (d, 1H), 4.1 (s, 3H), 2.6 (s, 3H).

### Example 9

### Synthesis of 3-Carboxy-4-methoxy phenylglyoxal pentafluorophenyl ester

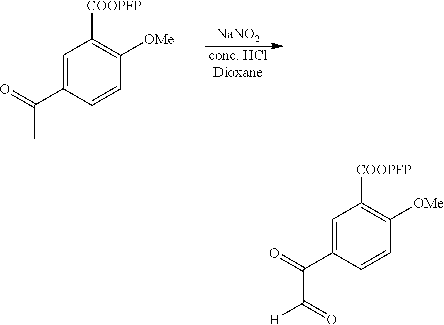

A suspension of 3-carboxy-4-methoxy acetophenone pentafluorophenyl ester (5 g, 0.014 mol) in a mixture of conc. HCl (4.7 mL) and 1,4-dioxane (15 mL) was heated to about 60° C. A solution of sodium nitrite (2.1 g, 0.030 mol) in water (6.1 mL) was added dropwise over a period of 4 h. The reaction was cooled and diluted with water (150 mL). The mixture was extracted with ethyl acetate (3×100 mL) and the combined extract was washed with water and brine. It was then dried (sodium sulfate) and concentrated. The crude product was purified by column chromatography (silica gel, 60-120 mesh; eluent: dichloromethane).

Yield: 1.0 g (19%). Purity was low (by TLC and NMR).

¹H NMR (DMSO-d₆) δ 8.8 (s, 1H), 8.4 (d, 1H), 7.4 (d, 1H), 5.9 (d, 1H), 4.0 (s, 3H).

See WO 93/17989, “Preparation of Substituted or Unsubstituted Phenylglyoxals.”

### Example 10

### Synthesis of Biotin TFP ester

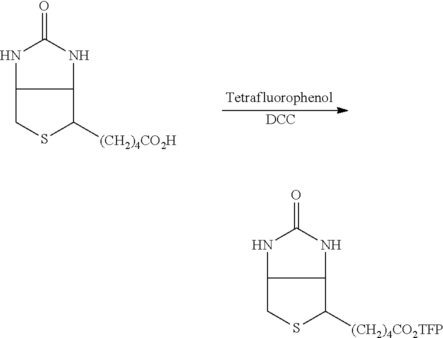

To a solution of biotin (10 g, 0.041 mol) in 1,4-dioxane (400 mL), tetrafluorophenol (9 g, 0.054 mol) was added. The mixture was cooled to about 5° C. and DCC (24 g, 0.11 mol) was added. The reaction mixture was stirred at RT for 48 h. After the completion of the reaction (by TLC), the solids were filtered off and the solvent removed under vacuum. The crude product was purified by column chromatography (silica gel, 60-120 mesh; eluent, chloroform:methanol, 9:1).

Yield: 7 g (42%).

¹H NMR (DMSO-d₆) δ 7.9 (m, 1H), 6.4 (d, 2H), 4.4 (m, 1H), 4.2 (m, 1H), 3.1 (m, 1H), 2.6-2.9 (m, 3H), 2.4 (m, 1H), 1.4-1.8 (m, 6H).

### Example 11

### Synthesis of N-(8-amino-3,6-dioxaoctanyl)biotinamide

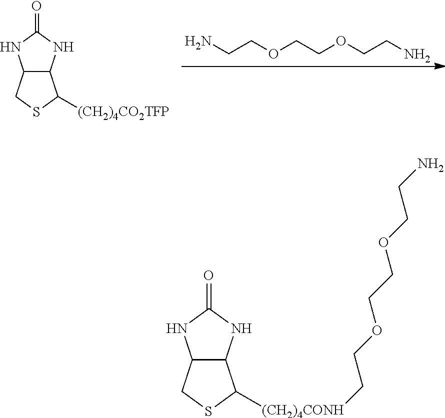

2,2′-(ethylenedioxy)bis(ethylamine) (21.1 g, 0.141 mol) was dissolved in dry acetonitrile (200 mL). Biotin TFP ester (2.8 g, 7.13 mmol) was dissolved in 450 mL dry acetonitrile at about 60° C. and added to the above solution after cooling to RT. The mixture was stirred at RT overnight. After the completion of the reaction (by TLC), the reaction mixture was concentrated and the residue was triturated with ether (300 mL) to afford a white solid. The crude product was further purified by column chromatography (silica gel, 60-120 mesh; eluent, chloroform:methanol, 2:8).

Yield: 2.5 g (95%).

¹H NMR (DMSO-d₆) δ 7.8 (t, 1H), 6.4 (d, 2H), 4.4 (m, 1H), 4.2 (m, 1H), 3.0-3.4 (m, 11H), 2.8 (dd, 1H), 2.6 (m, 2H), 2.5 (d, 1H), 2.1(t, 2H), 1.4 (m, 4H), 1.3 (m, 4H).

See Wilbur, D. S., Pathare, P. M., Hamlin, D. K., Weerawarna, S. A., Bioconjugate Chem. 1997, 8, 819-832.

### Example 12

### Synthesis of PMT Target 1

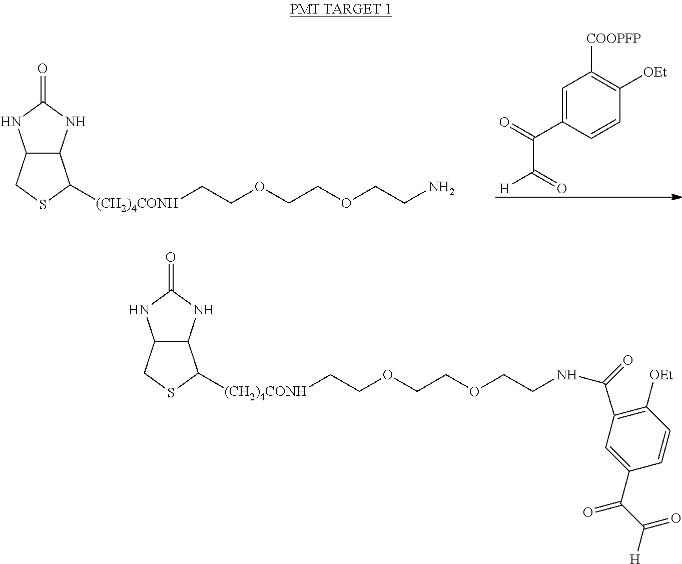

To a suspension of 3-carboxy-4-ethoxy phenylglyoxal PFP ester (1.0 g, 0.0025 mol) in dry acetonitrile (50 mL) a solution of N-(8-amino-3,6-dioxaoctanyl)biotinamide (0.9 g, 0.0024 mol) in dry methanol (30 mL) was added dropwise at about 5° C. The mixture was stirred for 30 min at about 5° C. The solvents were evaporated and crude product was purified by column chromatography (silica gel, 60-120 mesh; eluent, dichloromethane:methanol, 8.5:1.5).

Yield: 0.7 mg (50%).

¹H NMR (DMSO-d₆) δ 8.5 (s, 1H), 8.3 (t, 1H), 8.2(d, 1H), 7.7(t, 1H), 7.3(d, 1H), 6.4 (d, 2H), 5.4 (d, 1H), 4.3 (m, 3H), 4.1 (m, 1H), 3.0-3.4 (m, 11H), 2.8 (dd, 1H), 2.6 (m, 2H), 2.5 (d, 1H), 2.1(t, 2H), 1.2-1.8 (m, 11H).

¹³C NMR (DMSO-d₆) δ 192.68, 172.07, 164.0, 162.65, 160.06, 133.96, 132.46, 126.01, 122.59, 112.77, 96.54, 69.55, 69.16, 68.89, 65.03, 61.01, 59.17, 55.37, 54.04, 38.38, 35.06, 28.14, 28.00, 25.20, 14.21.

MS: M⁺ peak found (579).

### Example 13

### Synthesis of PMT Target 2

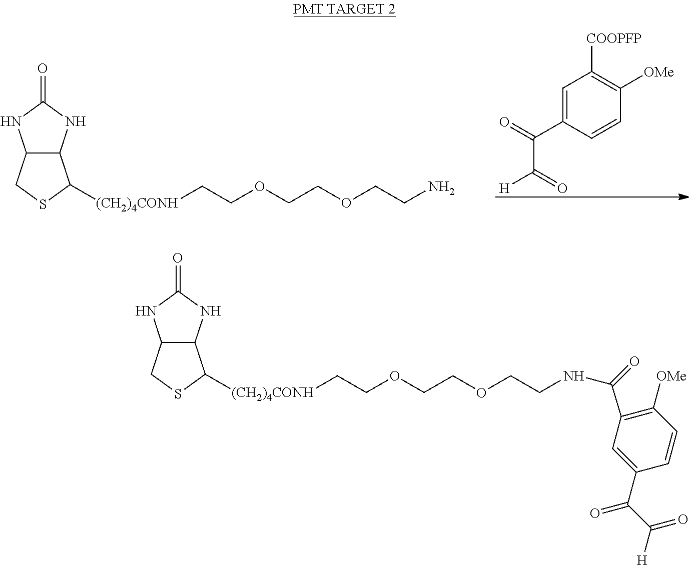

To a suspension of 3-carboxy-4-methoxy phenylglyoxal PFP ester (0.6 g, 0.0016 mol) in dry acetonitrile (20 mL) a solution of N-(8-amino-3,6-dioxaoctanyl)biotinamide (0.5 g, 0.0013 mol) in dry methanol (10 mL) was added dropwise at about 5° C. The mixture was stirred for 30 min at about 5° C. The solvents were evaporated and crude product was purified by column chromatography (silica gel, 60-120 mesh; eluent, dichloromethane:methanol, 8.5:1.5).

Yield: 0.3 mg (50%).

¹H NMR (DMSO-d₆): δ 8.5 (s, 1H), 8.3 (t, 1H), 8.2(d, 1H), 7.8(t, 1H), 7.2(d, 1H), 6.4 (d, 2H), 5.3 (s, 1H), 4.3 (m, 1H), 4.1 (m, 1H), 4.0 (s, 3H), 3.0-3.4 (m, 11H), 2.8 (dd, 1H), 2.6 (m, 2H), 2.5 (d, 1H), 2.1(t, 2H), 1.2-1.8 (m, 8H).

¹³C NMR (DMSO-d₆) δ 194.58, 192.76, 172.22, 164.22, 162.78, 160.76, 160.58, 133.99, 132.32, 126.11, 122.89, 112.04, 96.56, 89.32, 69.63, 69.20, 68.84, 61.09, 59.50, 55.44, 54.12, 38.47, 35.12, 28.21, 28.06, 25.27.

MS: (M+H)⁺ found (566).

NMR spectra were recorded in a “BRUKER AVANCE-300” (300 MHz) instrument and MS was recorded in a “VG-Mass lab Trio-2” quadruple system.

### Example 14

### Use of PMT Target 1 to Determine Angiotensin II

The eight amino acid sequence of Angiotensin II (Asp-Arg-Val-Tyr-Ile-His-Pro-Phe) contains one arginine residue, and thus can react with the PMT reagents of the invention that are specific for that amino acid residue, including those having a 1,2 dicarbonyl moiety as the amino acid reactive moiety. The reaction sequence between Angiotensin II and PMT Target 1 is shown below. As shown, the major product is the dehydrated adduct.

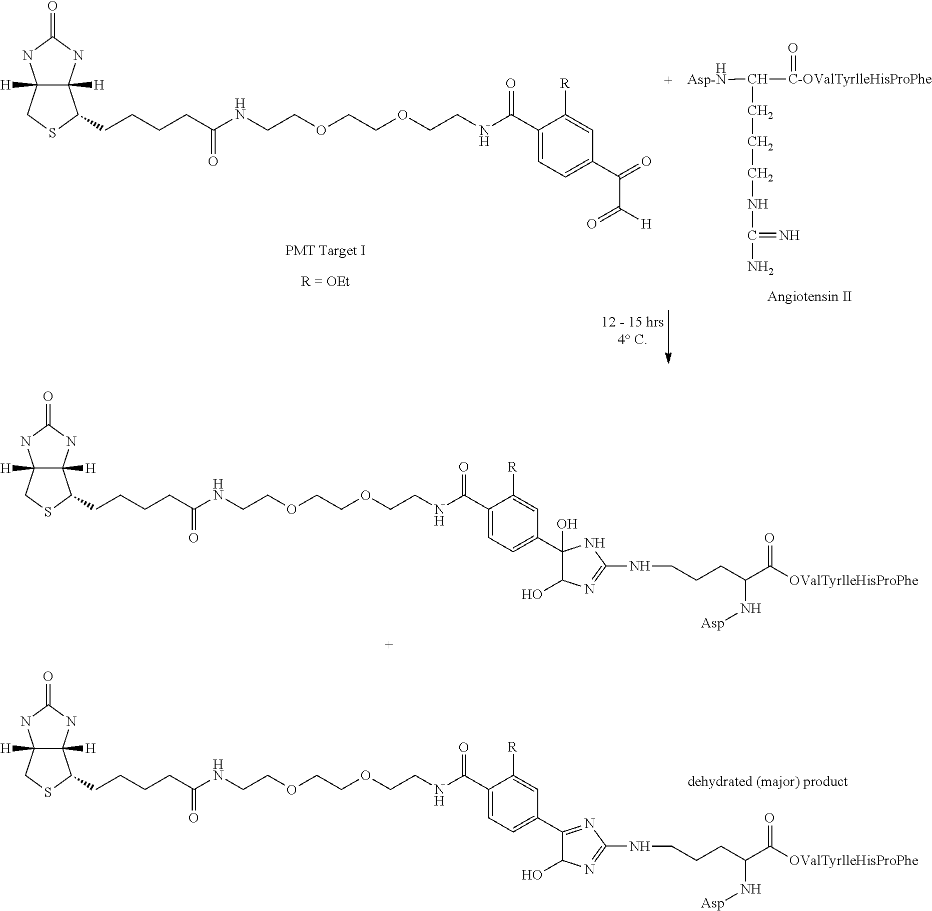

The PMT Target 1 (synthesized as outlined above) was dissolved in 4:1 sodium carbonate buffer (pH=11):DMSO, making a 100 mM solution. 10 μL of this solution was then added to 80 μL of carbonate buffer (pH=11) in an Eppendorf tube. To this mixture was added 10 μL of a 10 mM solution of Angiotensin II (1046.2 g/mol, Sigma) in deionized water. The reaction mixture is vortexed for 1 minute and then placed in a refrigerator at 4° C. for 12-15 hours. The reaction mixture was then desalted using ZipTip C-18 P10 (Millipore) and analyzed via MALDI using the following parameters.

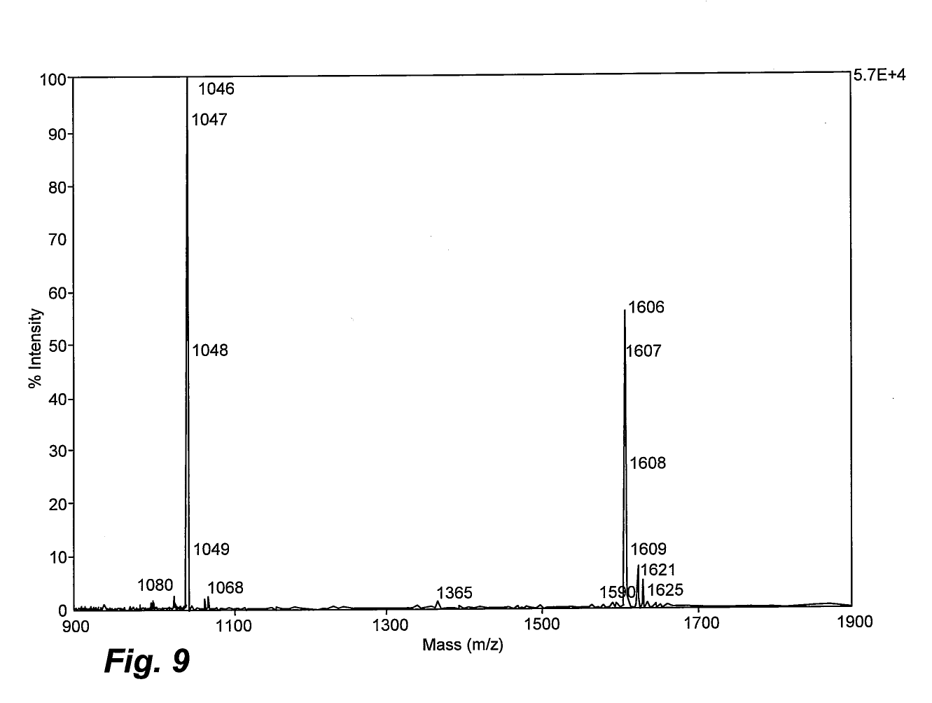

After desalting, dilutions can be made with deionized water (e.g., 1:10 or 1:100) in order to achieve the desired amount and concentration for analysis. Depending on the sensitivity of the MALDI instrument, a lesser amount of material is necessary and hence an increased dilution factor can be used. 0.5 μL of the reaction mixture was combined with 0.5 μL of 10 mg/mL α-cyano-4-hydroxycinnamic acid in 1:1 acetonitrile:water (0.1% trifluoroacetic acid) and spotted onto the MALDI plate. The spotted material was analyzed using angiotensin reflector mode (laser intensity ˜1200-2200). On the resulting spectrum, shown in FIG. 9, there are two main peaks. The second peak (at about 1606) represents the dehydrated adduct between the PMT Target 1 reagent and the Angiotensin II. The first peak (at about 1046) corresponds to unreacted Angiotensin II.

1. A protein mass tag (PMT) reagent for mass spectrometric analysis of proteins comprised of an amino acid reactive moiety that selectively reacts with certain protein functional groups, wherein said protein mass tag is differentially labeled with one or more non-isotopic chemical substituents.

2. The PMT reagent of claim 1, wherein said non-isotopic chemical substituents are selected from the group consisting of homologous organic substituents and halides.

3. The PMT reagent of claim 1, wherein said amino acid reactive moiety reacts with certain protein functional groups via a covalent reaction.

4. The PMT reagent of claim 3, wherein said protein functional group is an amino acid side chain.

5. The PMT reagent of claim 3, wherein said protein functional group is a post-translationally modified amino acid side chain.

6. The PMT reagent of claim 1, wherein said protein functional group is selected from the group consisting of an amino acid, a modified amino acid, a post-transitionally modified amino acid, a set of amino acids, a digested peptide or protein fragment.

7. The PMT reagent of claim 1, wherein said amino acid reactive moiety reacts with the guandinium group of arginine.

8. A plurality of PMT reagents for mass spectrometric analysis of proteins each comprised of an amino acid reactive moiety that selectively reacts with certain protein functional groups, wherein each of said PMT reagents is differently labeled with one or more non-isotopic chemical substituents.

9. The PMT reagent of claim 8 wherein said non-isotopic chemical substituents are selected from the group consisting of homologous organic substituents and halides.

10. The PMT reagent of claim 8 wherein said protein reactive moieties react with certain protein functional groups via covalent reactions.

11. The PMT reagent of claim 8 wherein said protein reactive moiety reacts with the side chain of arginine.

12. A plurality of PMT reagents for mass spectrometric analysis of proteins having the general formula:
RM-PRM
wherein RM is a recognition moiety and PRM is an amino acid reactive moiety that selectively reacts with certain protein functional groups, wherein each of said PMT reagents is differentially labeled with one or more non-isotopic chemical substituents.

13. The PMT reagents of claim 12 wherein RM is selected from the group consisting of biotin or an oligonucleotide having between 5 and 50 bases.

14. The PMT reagents of claim 12, wherein said non-isotopic chemical substituents are selected from the group consisting of homologous organic substituents and halides.

15. The PMT reagent of claim 12, wherein said amino acid reactive moiety reacts with certain protein functional groups via a covalent reaction.

16. The PMT reagent of claim 12, wherein said amino acid reactive moiety reacts with the guandinium group of arginine.

17. A plurality of PMT reagents for mass spectrometric analysis of proteins having the general formula:
RM-AM-PRM
wherein RM is a recognition moiety, AM is an accessory moiety and PRM is an amino acid reactive moiety that selectively reacts with certain protein functional groups, wherein each of said PMT reagents is differentially labeled with one or more non-isotopic chemical substituents.

18. The PMT reagents of claim 17 wherein RM is biotin and AM is a fluorescent compound.

19. A compound having the following formula: 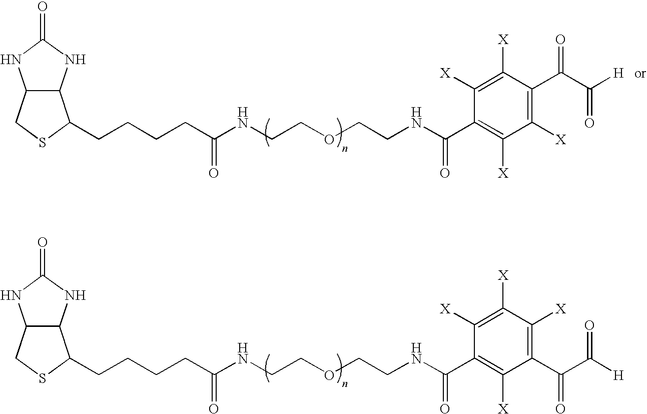
wherein
X is independently selected from the group consisting of H, D, OH, OD, R, OR, OSiR₃, Cl, Br, I, F, SH, SR, NH₂, NHR, and NR₂;
R is selected from the group consisting of an optionally substituted: C₁-C₂₀ alkyl, C₂-C₂₀ alkenyl, C₂-C₂₀ alkynyl, including deuterium substitutions; and
n=0-10

20. A compound having the following formula: 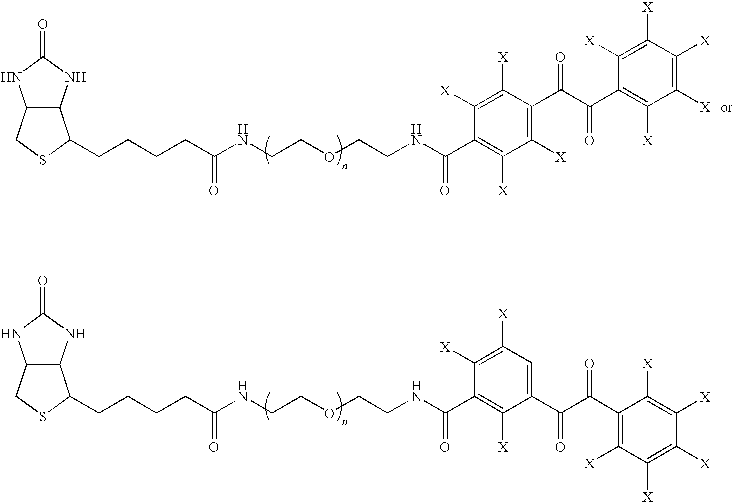
wherein
X is independently selected from the group consisting of H, D, OH, OD, R, OR, OSiR₃, Cl, Br, I, F, SH, SR, NH₂, NHR, and NR₂;
R is selected from the group consisting of an optionally substituted: C₁-C₂₀ alkyl, C₂-C₂₀ alkenyl, C₂-C₂₀ alkynyl, including deuterium substitutions; and
n=0-10.

21. A compound having the following formula:

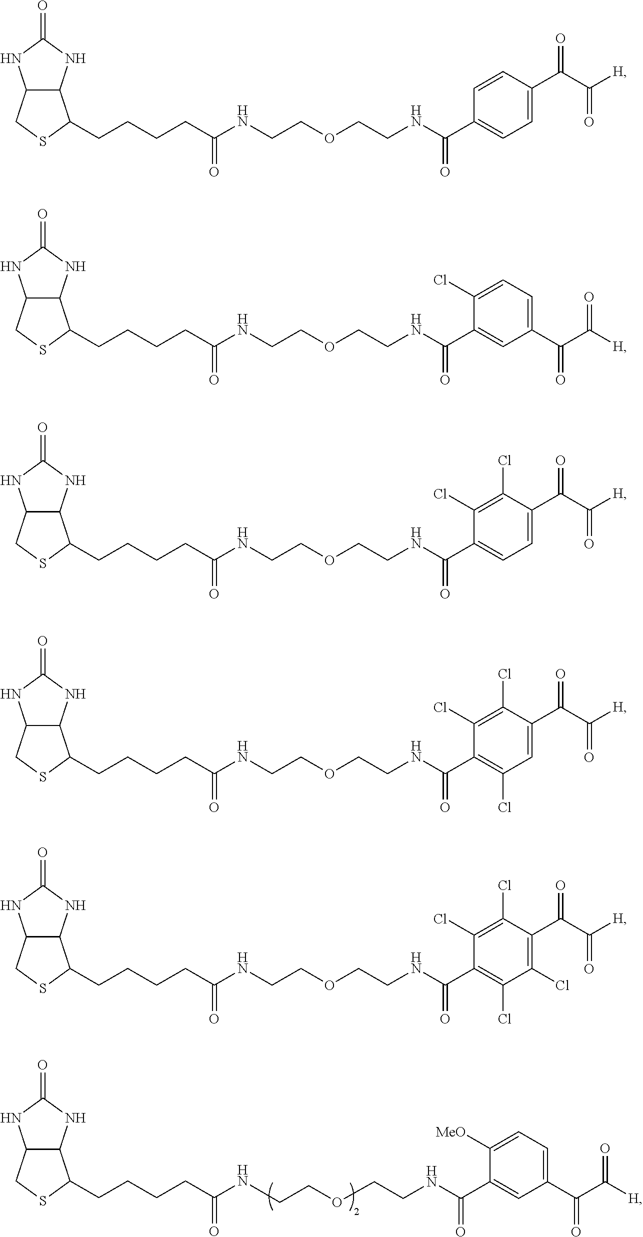

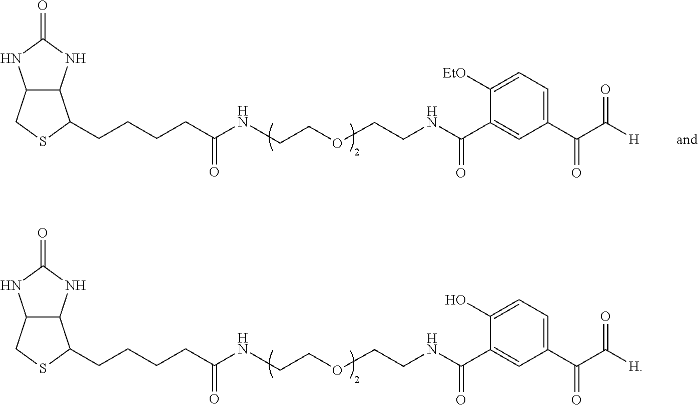

22. A compound having the formula:

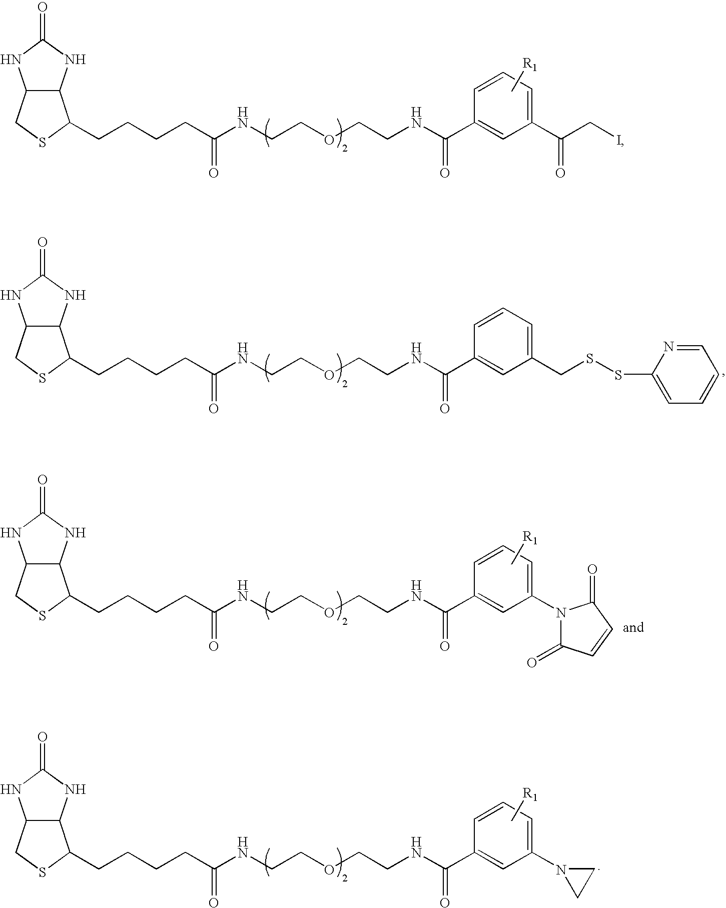

23. A method for identifying one or more proteins or protein components in one or more samples containing a mixture of proteins or protein components comprising:
a) providing a plurality of PMT reagents wherein each PMT reagent is comprised of an amino acid reactive moiety that selectively reacts with certain protein functional groups, wherein each of said PMT reagents is differently labeled with one or more non-isotopic chemical substituents;
b) contacting each sample with one of the PMT reagents to produce proteins or protein components in each sample labeled with a different PMT reagent;
c) isolating said labeled proteins or protein components; and
d) analyzing said labeled proteins or protein components.

24. The method of claim 23, further comprising digesting the proteins or protein components after containing the sample with the PMT reagents.

25. The method of claim 24 wherein the labeled proteins or protein components are analyzed by mass spectrometry.

26. A method for comparing two or more samples containing a mixture of one or more proteins or protein components comprising:
a) providing a plurality of PMT reagents wherein each PMT reagent is comprised of an amino acid reactive moiety that selectively reacts with certain protein functional groups, wherein each of said PMT reagents is differently labeled with one or more non-isotopic chemical substituents;
b) contacting each sample with a different PMT reagent to produce proteins or protein components in each sample labeled with a different PMT reagent;
c) isolating said labeled proteins or protein components; and
d) simultaneously analyzing said labeled proteins or protein components to quantitatively determine the relative amounts of protein or protein components in each sample.
